(19) United States
(12) Patent Application Publication
(10) Pub. No.: US 2022/0386992 A1
(43) Pub. Date: Dec. 8, 2022

(54) THERMOACOUSTIC IMAGING DEVICE WITH AN ACOUSTIC COUPLING PORTION

(71) Applicant: ENDRA Life Sciences Inc., Ann Arbor, MI (US)

(72) Inventors: Jordan Sengenberger, Ann Arbor, MI (US); Michael M. Thornton, London (CA)

(73) Assignee: ENDRA Life Sciences Inc., Ann Arbor, MI (US)

(21) Appl. No.: 17/340,058

(22) Filed: Jun. 6, 2021

(51) Int. Cl. A61B 8/00; G10K 11/02

(52) U.S. Cl. CPC A61B 8/4444; G10K 11/02; A61B 8/4272; G01K 11/22

(57) ABSTRACT

A thermoacoustic imaging device for coupling to a region of interest on a patient is disclosed. The device includes a housing having a surface, wherein the surface comprises an acoustic coupling portion having a substantially perpendicular extent relative to the surface. In one embodiment, the perpendicular extent extends to the surface. In one embodiment, the perpendicular extent extends to the surface and the outwardly from the surface. In one embodiment, the perpendicular extent extends only from the surface.

### FIELD

The subject disclosure relates to thermoacoustic imaging. In particular, the disclosure describes a thermoacoustic imaging device with an integrated thermoacoustic transducer coupling portion.

### BACKGROUND

Thermoacoustic imaging is an imaging modality that provides information relating to the thermoelastic properties of tissue. Thermoacoustic imaging uses short pulses of electromagnetic energy, such as radio frequency (RF) pulses, directed into a subject to heat absorbing features within the subject rapidly, which in turn induces thermoacoustic pressure waves that are detected using acoustic receivers, such as one or more thermoacoustic or ultrasound transducer arrays. The detected thermoacoustic pressure waves are analyzed through signal processing and processed for presentation as thermoacoustic images that can be interpreted by an operator.

The RF pulses can impact the one or more thermoacoustic or ultrasound transducer arrays, resulting in signal noise and signal artifacts. The signal noise and signal artifacts can negatively affect signal processing, resulting in degraded thermoacoustic images. Hence, the effect of RF pulses on the one or more thermoacoustic or ultrasound transducer arrays should be mitigated.

While mitigating the effect of RF pulses is one goal, it is also important to maximize thermoacoustic signal strength (amplitude).

Hence, there exists a need to provide a device that mitigates the effect of RF pulses while maximizing thermoacoustic signal strength.

### SUMMARY

A thermoacoustic imaging device for coupling to a region of interest on a patient is disclosed. The device includes a housing having a surface, a portion of the surface having a substantially planar portion for coupling to the region of interest, wherein the surface comprises an acoustic coupling portion having a substantially perpendicular extent relative to the surface.

In one embodiment, the perpendicular extent extends to the surface. In one embodiment, the perpendicular extent extends to the surface and the outwardly from the surface. In one embodiment, the perpendicular extent extends only from the surface.

In one embodiment, a device for coupling to a region of interest comprises: a radio-frequency emitter comprising a waveguide with a radio-frequency source, radio-frequency emitter sides, and an exit window; a thermoacoustic transducer comprising a signal cable, thermoacoustic transducer sides, and a front face; a housing configured to contain the radio-frequency emitter and thermoacoustic transducer, wherein the housing enables the exit window and front face to be held flush with respect to each other; and an acoustic coupler member comprising a standoff that is attached to and covers the front face of the thermoacoustic transducer, wherein the acoustic coupler member is configured to be coupled to the region of interest.

In one embodiment, the acoustic coupler member has an electrical impedance outside of a range of 2 kilo-ohms to 5 kilo-ohms when the radio-frequency emitter provides pulsed radio-frequency energy between a range of 13 MHz and 2.54 Ghz.

In one embodiment, a speed of sound in the acoustic coupler member is between 1520 and 1560 meters per second.

It should be appreciated that this Summary is provided to introduce a selection of concepts in a simplified form that are further described below in the Detailed Description. This Summary is not intended to be used to limit the scope of the claimed subject matter.

### BRIEF DESCRIPTION OF THE DRAWINGS

Embodiments will now be described more fully with reference to the accompanying drawings in which:

FIGS. 1A and 1B schematically show a thermoacoustic probe with a stand-off and set-back.

FIGS. 2A and 2B schematically show a thermoacoustic probe with a set-back.

FIGS. 3A and 3B schematically show a thermoacoustic probe with a stand-off.

FIGS. 4A and 4B schematically show a thermoacoustic transducer with a stand-off.

FIG. 5 shows a perspective view of an exemplary thermoacoustic probe with a stand-off.

FIG. 6 shows a perspective view of an exemplary thermoacoustic probe without a stand-off or set-back.

### DETAILED DESCRIPTION OF THE EMBODIMENTS

The foregoing summary, as well as the following detailed description of certain examples will be better understood when read in conjunction with the appended drawings. As used herein, an element or feature introduced in the singular and preceded by the word “a” or “an” should be understood as not necessarily excluding the plural of the elements or features. Further, references to “one example” or “one embodiment” are not intended to be interpreted as excluding the existence of additional examples or embodiments that also incorporate the described elements or features. Moreover, unless explicitly stated to the contrary, examples or embodiments “comprising” or “having” or “including” an element or feature or a plurality of elements or features having a particular property may include additional elements or features not having that property. Also, it will be appreciated that the terms “comprises”, “has”, “includes” means “including but not limited to” and the terms “comprising”, “having” and “including” have equivalent meanings.

As used herein, the term “and/or” can include any and all combinations of one or more of the associated listed elements or features.

It will be understood that when an element or feature is referred to as being “on”, “attached” to, “connected” to, “coupled” with, “contacting”, etc. another element or feature, that element or feature can be directly on, attached to, connected to, coupled with or contacting the other element or feature or intervening elements may also be present. In contrast, when an element or feature is referred to as being, for example, “directly on”, “directly attached” to, “directly connected” to, “directly coupled” with or “directly contacting” another element of feature, there are no intervening elements or features present.

It will be understood that spatially relative terms, such as “under”, “below”, “lower”, “over”, “above”, “upper”, “front”, “back” and the like, may be used herein for ease of description to describe the relationship of an element or feature to another element or feature as illustrated in the figures. The spatially relative terms can however, encompass different orientations in use or operation in addition to the orientations depicted in the figures.

In one embodiment, the RF emitter has a frequency between about 10 MHz and 100 GHz and has a pulse duration between about 0.1 nanoseconds and 10 microseconds.

For the purposes of this disclosure, a thermoacoustic transducer is an ultrasound transducer that is configured to receive thermoacoustic signals and convert the thermoacoustic signals to electrical signals. In one embodiment, the ultrasound transducer can both transmit and receive ultrasound, and receive thermoacoustic signals. In a separate embodiment, the ultrasound transducer is configured to only receive ultrasound and thermoacoustic signals.

An objective discussed in this disclosure is minimizing acoustic signal artifacts caused by radio-frequency emission. One source of acoustic signal artifacts is caused by radio-frequency emissions from the tissue being monitored at the surface of the thermoacoustic transducer. This source can be minimized in two ways: first, create an electrical impedance mismatch between the tissue being monitored and the thermoacoustic transducer (which can be in direct contact with the monitored tissue or have an intermediary such as a gel between the monitored tissue and thermoacoustic transducer); second, create enough distance between the thermoacoustic transducer (which is electrically sensitive) to prevent a spark (electrical short circuit) from jumping the distance between said tissue and the thermoacoustic transducer. Preventing the spark (electrical short circuit) then minimizes electrical noise (artifacts) to an amount proportional to the inverse squared of the distance between said tissue and the thermoacoustic transducer.

Another objective discussed in this disclosure is maintaining acoustic transmissivity (compatibility). Hence, it's an objective to maximize the thermoacoustic signal that the thermoacoustic transducer receives, while minimizing electro-magnetic interference (EMI).

In conjunction with electro-magnetic shielding, the disclosure herein contemplates two techniques: a set-back technique and a stand-off technique. Each technique is effective because they reduce the magnitude of the RF energy, prior to the RF energy reaching the thermoacoustic transducer electronics. The effect of the set-back technique and stand-off technique is an unexpected result, because the distance is minor while the effect is substantial.

Prior thermoacoustic probe embodiments had the RF emitter and thermoacoustic transducer flush with each other. The present disclosure describes two techniques: (A) the thermoacoustic transducer is set-back (moved back) from the RF emitter; (B) the thermoacoustic transducer is in a standoff position (moved forward) from the RF emitter.

Acoustic impedance (Z) is a physical property of tissue. It describes how much resistance an ultrasound, acoustic, or thermoacoustic signal encounters as it passes through a tissue. The SI unit for acoustic impedance is the Rayl, kg/(m2s), after J W Strutt, 3rd Baron Rayleigh. Acoustic impedance depends upon: (1) the density of the tissue (d, in kg/m3); and (2) the speed of the sound wave (c, in m/s). This relation may be expressed by Z=d×c (density multiplied by speed of sound in the tissue).

So, if the density of a tissue increases, impedance increases. Similarly, but less intuitively, if the speed of sound increases, then impedance also increases.

The effect of acoustic impedance in medical ultrasound and thermoacoustics becomes noticeable at interfaces between different tissue types. The ability of an ultrasound or thermoacoustic wave to transfer from one tissue type to another depends on the difference in impedance of the two tissues. If the difference is large, then the sound is reflected. We grasp this intuitively at a macroscopic level. If you were to yell into a canyon, you would expect an echo to return to you. The sound wave in air meets the dense rocky canyon wall and reverberates off of it back to you; the sound wave does not just pass into the rock. This is due to the difference in impedance.

Similarly, when an ultrasound or thermoacoustic signal passes through muscle tissue and encounters bone, it reflects off of it due to the difference in density between the tissues.

The amount of reflection that occurs in a perpendicular direction can be expressed by:

Reflection fraction=[(Z2−Z1)/(Z2+Z1)]² Eq. (1)

Where Z1 and Z2 represent the impedance in tissue 1 and tissue 2, respectively. Because you generally cannot reflect more sound than you originally sent, the (Z2−Z1) term must be the numerator.

Examples of impedance for bodily tissues (in kg/(m2s)): air 0.0004×10⁶; lung 0.18×10⁶; fat 1.34×10⁶; water 1.48×10⁶; kidney 1.63×10⁶; blood 1.65×10⁶; liver 1.65×10⁶; muscle 1.71×10⁶; bone 7.8×10⁶. For example, using these values with the equation (1) above, in an exemplary patient, it is apparent that less than 1% of sound is reflected at a fat-liver interface.

If the sound wave is not perpendicular to a surface, some of the sound wave will be reflected away from the transducer. This reduces the amount of signal strength available for a thermoacoustic transducer.

Electrical impedance is the opposition by a system to the flow of energy from an electrical source. For constant signals, this impedance can also be constant. For varying signals, the impedance will vary with the signal frequency. Electrical impedance, like electrical resistance, is measured in ohms. In general, electrical impedance has a complex value; this means that loads generally have a resistance component (symbol: R) which forms the real part of Z and a reactance component (symbol: X) which forms the imaginary part of Z.

In simple cases (such as low-frequency or direct-current power transmission) the reactance may be negligible or zero. Hence, the impedance in simple cases can be considered a pure resistance, expressed as a real number. In the following summary we will consider the general case when resistance and reactance are both significant, and the special case in which the reactance is negligible.

Reflection-less matching: Electrical impedance matching to minimize reflections is achieved by making the load impedance equal to the source impedance. If the source impedance, load impedance and transmission line characteristic impedance are purely resistive, then reflection-less matching is the same as maximum power transfer matching.

Maximum power transfer matching: complex conjugate matching is used when maximum power transfer is required. That is, when a load impedance is a source impedance with the complex conjugate. A conjugate match is different from a reflection-less match when either the source or load has a reactive component.

If the source has a reactive component, but the load is purely resistive, then matching can be achieved by adding a reactance of the same magnitude but opposite sign to the load. This simple matching network, consisting of a single element, will usually achieve a perfect match at only a single frequency. This is because the added element will either be a capacitor or an inductor, whose impedance in both cases is frequency dependent, and will not, in general, follow the frequency dependence of the source impedance. For wide bandwidth applications, a more complex network can be designed.

Power transfer: whenever a source of power with a fixed output impedance such as an electric signal source, a radio transmitter or a mechanical sound (e.g., a loudspeaker) operates into a load, the maximum possible power is delivered to the load when the impedance of the load (load impedance or input impedance) is equal to the complex conjugate of the impedance of the source (that is, its internal impedance or output impedance). For two impedances to be complex conjugates their resistances must be equal, and their reactances must be equal in magnitude but of opposite signs. In low-frequency or DC systems (or systems with purely resistive sources and loads) the reactances are zero, or small enough to be ignored. In this case, maximum power transfer occurs when the resistance of the load is equal to the resistance of the source.

Impedance matching is not always necessary. For example, if a source with a low impedance is connected to a load with a high impedance the power that can pass through the connection is limited by the higher impedance. This maximum-voltage connection is a common configuration called impedance bridging or voltage bridging, and is widely used in signal processing. In such applications, delivering a high voltage (to minimize signal degradation during transmission or to consume less power by reducing currents) is often more important than maximum power transfer. Strictly speaking, impedance matching only applies when both source and load devices are linear; however, matching may be obtained between nonlinear devices within certain operating ranges.

The impedance of free space, Z₀, is a physical constant relating the magnitudes of the electric and magnetic fields of electromagnetic radiation travelling through free space, which may be expressed as: Z₀=|E|/|H|, where |E| is the electric field strength and |H‥ the magnetic field strength. Its presently accepted value is:

Z₀=376.730313668Ω Eq. (2)

The impedance of free space (that is the wave impedance of a plane wave in free space) is equal to the product of the vacuum permeability μ₀ and the speed of light in vacuum c₀. Before 2019, the values of both these constants were taken to be exact (they were given in the definitions of the ampere and the metre respectively), and the value of the impedance of free space was therefore likewise taken to be exact. However, with the redefinition of the SI base units that came into force on 20 May 2019, the impedance of free space is subject to experimental measurement because only the speed of light in vacuum c₀ retains an exactly defined value.

For some embodiments discussed in the present disclosure, it is an object to minimize the acoustic reflection fraction by substantially matching the acoustic impedance of tissue with traversed materials of the thermoacoustic device 100, which may be a probe, while maximizing the electro-magnetic reflection (i.e., minimizing electrical power transfer). This matching occurs by creating an electrical impedance mismatch between the tissue being monitored and the thermoacoustic transducer.

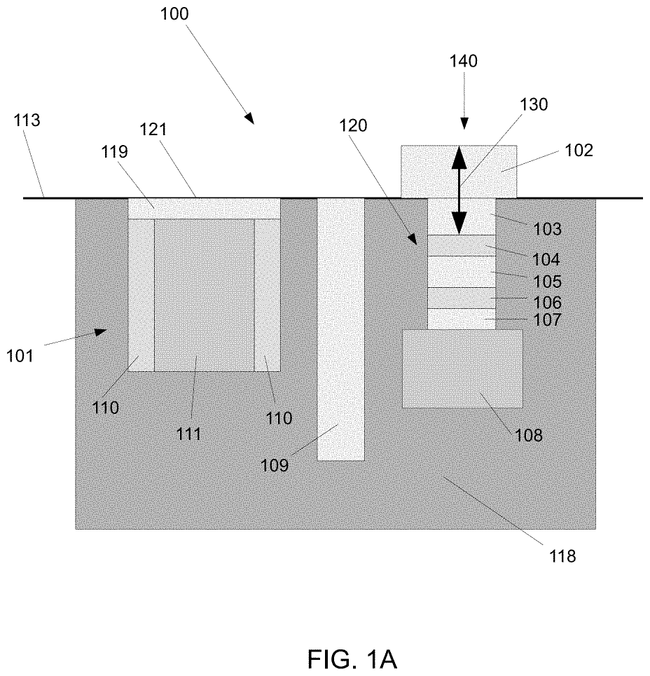

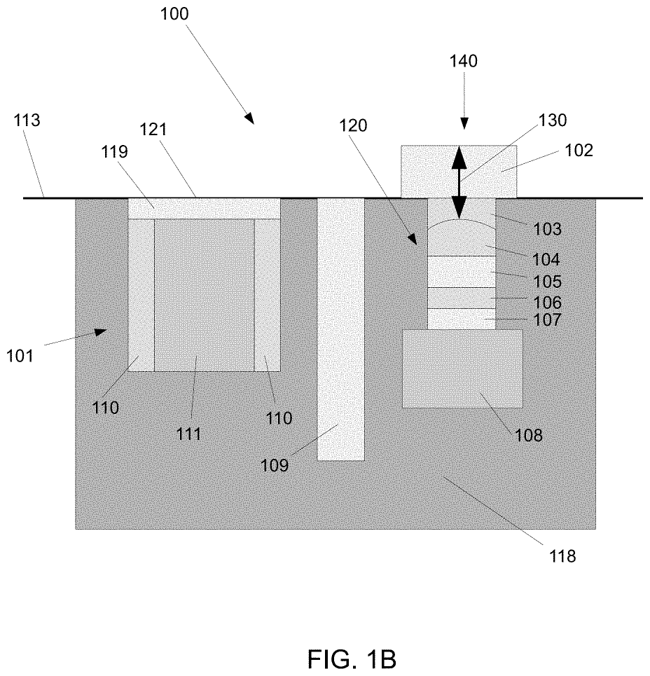

FIGS. 1A and 1B schematically shows a thermoacoustic device 100 having an acoustic coupling portion 140 which includes both an exemplary stand-off portion 102 and an exemplary set-back portion 103. As FIG. 1A shows, the thermoacoustic device 100 includes a radio-frequency (RF) emitter 101, the stand-off portion 102, the set-back portion 103, a thermoacoustic transducer surface 104, i.e., a front face, a thermoacoustic transducer matching layer 105, a piezo-electric crystal 106, a piezo-electric backing 107, a RF shield box 108, a acoustic isolation 109, a RF waveguide 110, a RF insert 111, a thermoacoustic probe housing 118, a RF matching layer 119, and a thermoacoustic transducer 120. In one embodiment, an exit window 121 is coupled to the RF matching layer 119 and is flush with the surface 113. The exit window 121 may extend from edges of the RF matching layer 119 and/or be sized and adapted to align with the RF matching layer 119. In one embodiment, the thermoacoustic transducer surface 104 is substantially planar. In one embodiment, the thermoacoustic transducer surface 104 is curved or includes a curved portion.

The RF emitter 101 includes the RF waveguide 110, the RF insert 111, and the RF matching layer 119.

The thermoacoustic transducer 120 includes the thermoacoustic transducer surface 104, the thermoacoustic transducer matching layer 105, the piezo-electric crystal 106, the piezo-electric backing 107, and the RF shield box 108 which protects thermoacoustic electronics (not shown). The piezo-electric backing 107 is configured to selectively transfer electric signals to the thermoacoustic electronics.

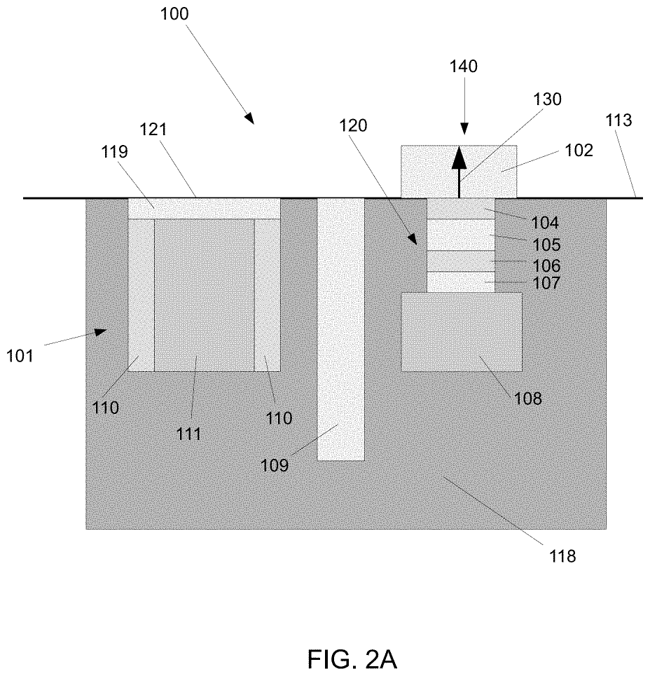

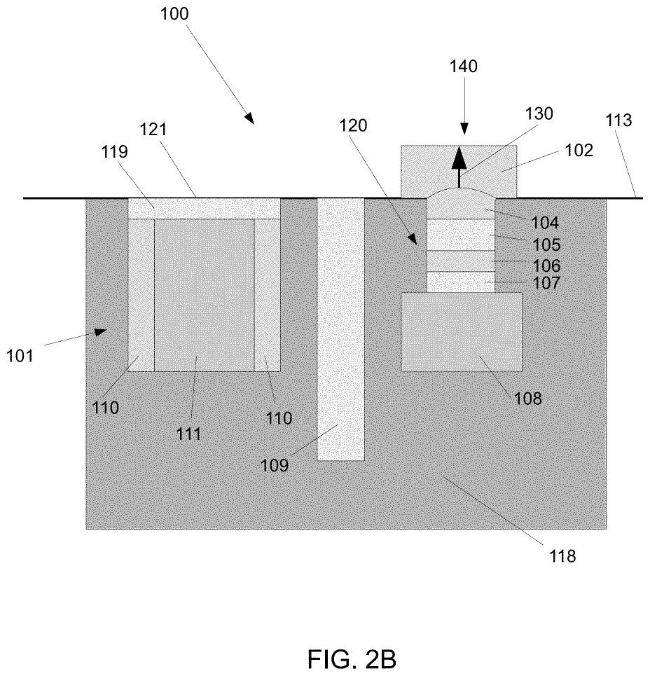

The thermoacoustic housing 118 holds the RF emitter 101, the thermoacoustic transducer 120, and the acoustic isolation 109. The thermoacoustic housing 118 also holds the set-back portion 103 as in the embodiments shown in FIGS. 1A-1B and FIGS. 2A-2B.

The thermoacoustic device 100 includes a surface 113 that may be formed by the housing 118. This surface 113 can be used to contact tissue directly, or through a gel as described herein above. In one embodiment, the surface 113 is flush. In one embodiment, the surface 113 is used as a reference plane for the RF emitter 101 and the thermoacoustic transducer 120.

In various embodiments, the acoustic coupling portion 140, which may be formed of either of both of the stand-off portion 102 and the set-back portion 103, is formed of materials configured to maximize acoustic signal transfer and minimize electrical noise created by signals from the RF emitter 101. As described hereinabove, maximizing acoustic signal transfer also minimizes the acoustic reflection. Hence, it is preferably, in many embodiments, that the acoustic properties of the stand-off portion 102 and the set-back portion 103 substantially match tissue acoustic properties. The electrical impedance properties of the stand-off portion 102 and the set-back portion 103 are preferably chosen to mismatch tissue electrical impedance properties.

In various embodiments, the stand-off portion 102 and/or set-back portion 103 may be formed of materials having substantially the same acoustic impedance properties as tissue which may be achieved using silicon, silicon-based elastomers, silicon-based polymers, silicon-based rubber, rubber, or other silicon-based materials, and any combination thereof. In one embodiment, the material of construction used for the stand-off portion 102 and/or the set-back portion 103 is RTV 630™. In one embodiment, the material of construction used for the stand-off portion 102 and/or the set-back portion 103 is SILGARD 184™. In one embodiment, the material of construction used for the stand-off portion 102 and the set-back portion 103 has an acoustic impedance between 1.0×10⁶ kg/(m2s) and 2.0×10⁶ kg/(m2s).

In one embodiment, the material of construction used for the stand-off portion 102 and the set-back portion 103 has a relative permittivity between 2.0 and 4.0. For comparison, air has a relative permittivity of 1.0 and tissue has a relative permittivity of roughly 50. For the purposes of this disclosure, assume that tissue has a relative permittivity between 40 and 60.

The acoustic coupling portion 140, which may be the stand-off portion 102 and/or the set-back portion 103, has a perpendicular extent 130 relative to an exterior surface 113 of the housing 118. The acoustic coupling portion 140 extends inwardly or outwardly from the planar surface 113 substantially perpendicularly. A stand-off portion 102 has a perpendicular extent 130 that extends outwardly from the surface 113. A set-back portion 103 has a perpendicular extent 130 that extends inwardly from the surface 113. The set-back portion 103 may be formed from a material, as described hereinabove, or may be a void space which may be sized and shaped to receive a gel during use. The expression “substantially perpendicularly” is intended here to define, in particular, an alignment of a direction relative to a reference direction, the direction and the reference direction, in particular as viewed in one plane, enclosing an angle of 90°, and the angle having a maximum deviation of, in particular, less than 10-degrees, advantageously less than 5-degrees, and particularly advantageously less than 2-degrees.

The acoustic coupling portion 140 may be formed of any number of shapes and sizes, adapted to traversing acoustic energy therethrough, including, e.g., square, circular, elliptical, etc. in form.

The acoustic coupling portion 140 shown in FIG. 1A is formed of the stand-off portion 102 having a perpendicular extent 130 that extends outwardly from the planar surface 113 and the set-back portion 103 having a perpendicular extent 130 that extends inwardly from the planar surface 113. In one embodiment, the stand-off portion 102 and set-back portion 103 are combined as one element and may be integrally formed.

In one embodiment, thermoacoustic device 100 includes a radio-frequency emitter 101 having an exit window 121, a thermoacoustic transducer 120 comprising a front face 104 for receiving reflected acoustic energy, a housing 118 configured to contain the thermoacoustic transducer 120 and the radio-frequency emitter 101, wherein the housing 118 comprises a surface 113, a portion of the surface having a substantially planar portion for coupling to the region of interest, wherein the exit window 121 of the radio-frequency emitter 101 and front face 104 of the thermoacoustic transducer 120 are aligned flush with the surface 113, and wherein the surface 113 comprises an acoustic coupling portion 140 having a substantially perpendicular extent relative to the front face 104 of the thermoacoustic transducer 120.

FIG. 1B schematically shows an exemplary thermoacoustic device 100 including the thermoacoustic transducer surface 104 with a curved surface. The set-back portion 103 may be sized and adapted to mirror the curved surface, i.e., the thermoacoustic transducer surface 104 is formed with a convex curved surface, while the set-back portion 103 is formed with an opposing concaved surface to match substantially thereon. As FIG. 1B shows, the acoustic coupling portion 140, which includes the stand-off portion 102 and/or the set-back portion 103, has a perpendicular extent 130 relative to an exterior surface 113 of the housing 118. The acoustic coupling portion 140 extends inwardly or outwardly from the planar surface 113 substantially perpendicularly. The stand-off portion 102 has a perpendicular extent 130 that extends outwardly from the surface 113. The set-back portion 103 has a perpendicular extent 130 that extends inwardly from the surface 113 and terminates at the curved surface of the thermoacoustic transducer surface 104.

FIG. 2A schematically shows an exemplary thermoacoustic device 100 having an exemplary acoustic coupling portion 140 formed of a set-back portion 103 having a perpendicular extent 130 that extends inwardly from the planar surface 113. Shown are the thermoacoustic device 100, the radio-frequency (RF) emitter 101, the set-back portion 103, the thermoacoustic transducer surface 104, the thermoacoustic transducer matching layer 105, the piezo-electric crystal 106, the piezo-electric backing 107, the RF shielding 108, the acoustic isolation 109, the RF waveguide 110, the RF insert 111, representation of flush alignment 113, and the thermoacoustic housing 118.

FIG. 2B schematically shows an exemplary thermoacoustic device 100 including the thermoacoustic transducer surface 104 with a curved surface. The stand-off portion 102 may be sized and adapted to mirror the curved surface, i.e., the thermoacoustic transducer surface 104 is formed with a convex curved surface, while the stand-off portion 102 is formed with an opposing concaved surface to match substantially thereon. As FIG. 2B shows, the acoustic coupling portion 140, which includes the stand-off portion 102, has a perpendicular extent 130 relative to the curved surface of the thermoacoustic transducer surface 104. The acoustic coupling portion 140 extends outwardly from the curved surface of the thermoacoustic transducer surface 104 substantially perpendicularly. The stand-off portion 102 has a perpendicular extent 130 that extends outwardly from the curved surface of the thermoacoustic transducer surface 104 substantially perpendicularly. In one embodiment, the perpendicular extent 130 may be defined as the length from a peak of the curved surface of the thermoacoustic transducer surface 104 to a top of the stand-off portion 102.

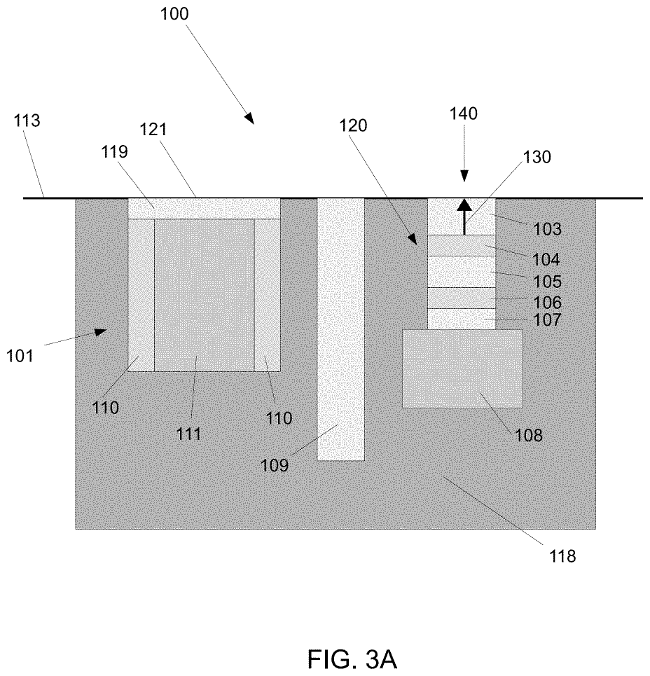

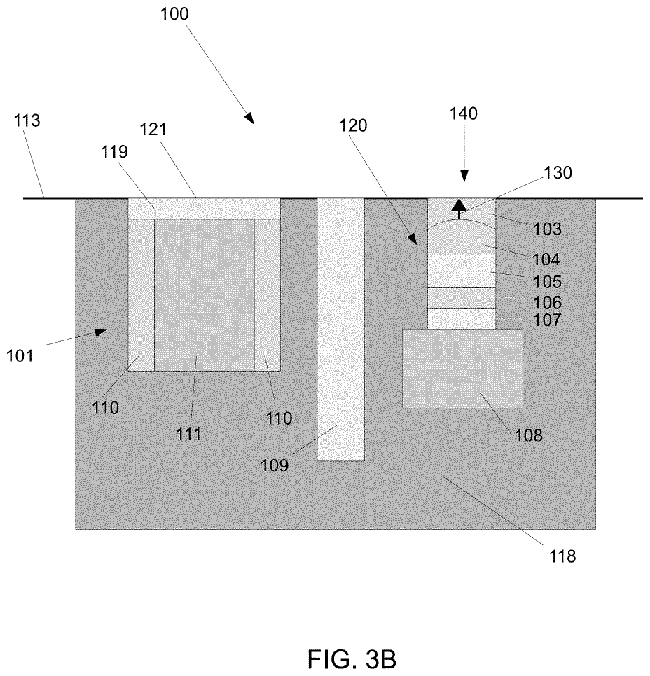

FIG. 3A schematically shows an exemplary thermoacoustic device 100 having an exemplary acoustic coupling portion 140 formed of a stand-off portion 102 having a perpendicular extent 130 that extends outwardly from the planar surface 113. Shown are the thermoacoustic device 100, the radio-frequency (RF) emitter 101, the stand-off portion 102, the thermoacoustic transducer surface 104, the thermoacoustic transducer matching layer 105, the piezo-electric crystal 106, the piezo-electric backing 107, the RF shielding 108, the acoustic isolation 109, the RF waveguide 110, the RF insert 111, and the thermoacoustic housing 118.

FIG. 3B schematically shows an exemplary thermoacoustic device 100 including the thermoacoustic transducer surface 104 with a curved surface. The set-back portion 103 may be sized and adapted to mirror the curved surface, i.e., the thermoacoustic transducer surface 104 is formed with a convex curved surface, while the set-back portion 103 is formed with an opposing concaved surface to match substantially thereon. As FIG. 3B shows, the acoustic coupling portion 140, which includes the set-back portion 103, has a perpendicular extent 130 relative to the curved surface of the thermoacoustic transducer surface 104. The acoustic coupling portion 140 extends outwardly from the curved surface of the thermoacoustic transducer surface 104 substantially perpendicularly. The set-back portion 103 has a perpendicular extent 130 that extends outwardly from the curved surface of the thermoacoustic transducer surface 104 substantially perpendicularly. In one embodiment, the perpendicular extent 130 may be defined as the length from a peak of the curved surface of the thermoacoustic transducer surface 104 to a top of the set-back portion 103.

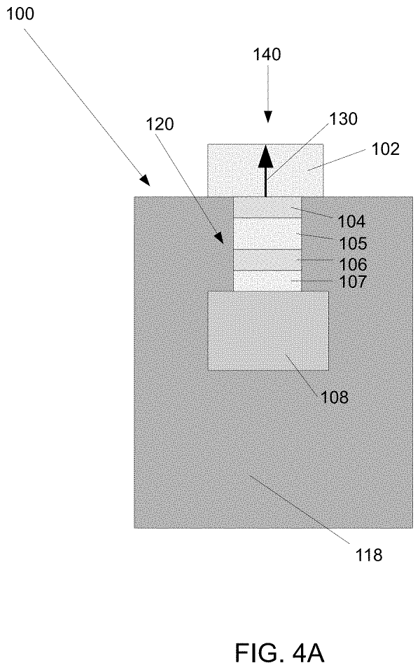

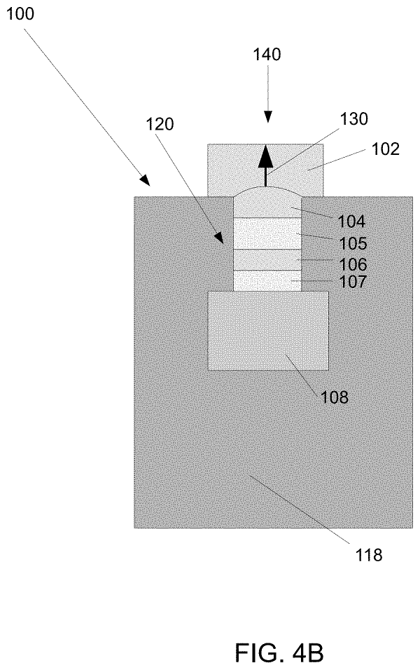

FIG. 4A schematically shows a thermoacoustic transducer 120 having an exemplary acoustic coupling portion 140 formed of a stand-off portion 102 having a perpendicular extent 130 that extends outwardly from the planar surface 113. Shown are the thermoacoustic device 100, the stand-off portion 102, the set-back portion 103, the thermoacoustic transducer surface 104, the thermoacoustic transducer matching layer 105, the piezo-electric crystal 106, the piezo-electric backing 107, the RF shielding 108, the acoustic isolation 109, and the thermoacoustic housing 118.

FIG. 4B schematically shows an exemplary thermoacoustic device 100 including the stand-off portion 102 with a curved surface. In this embodiment, the stand-off portion 102 can be used to contact tissue directly, or through a gel as described herein above. In one embodiment, the stand-off portion 102 or the top curved surface thereof is used as a reference for the thermoacoustic transducer 120. As FIG. 4B shows, the acoustic coupling portion 140, which includes the stand-off portion 102, has a perpendicular extent 130 relative to the thermoacoustic transducer surface 104. The acoustic coupling portion 140 extends outwardly from the thermoacoustic transducer surface 104 substantially perpendicularly. The stand-off portion 102 has a perpendicular extent 130 that extends outwardly from the thermoacoustic transducer surface 104 substantially perpendicularly. In one embodiment, the perpendicular extent 130 may be defined as the length from the thermoacoustic transducer surface 104 to a peak of the stand-off portion 102.

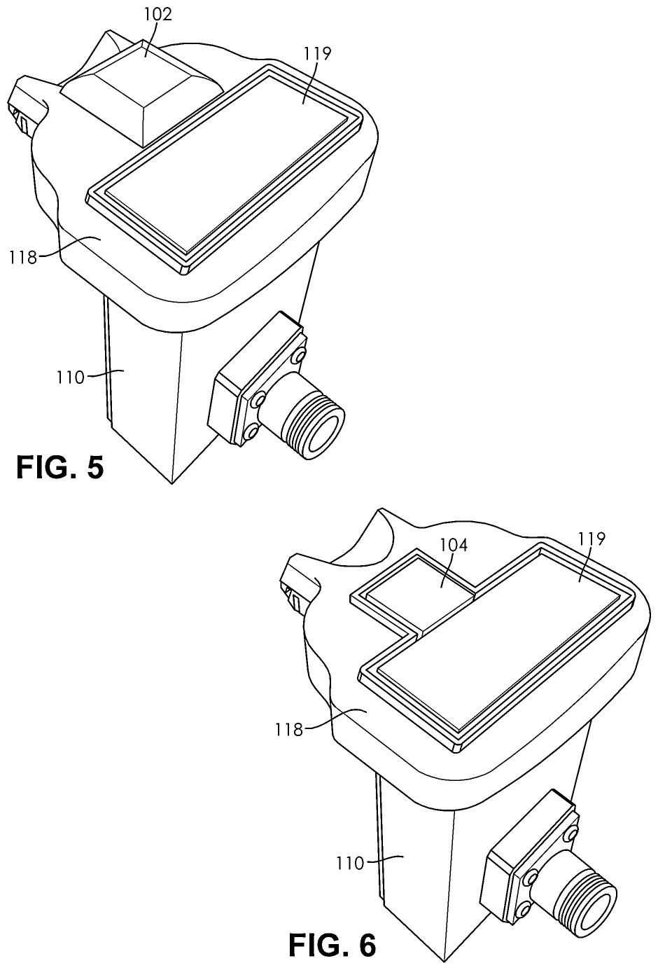

FIG. 5 shows a perspective view of a thermoacoustic device 100 having a stand-off portion 102. Shown are the thermoacoustic device 100, the radio-frequency (RF) emitter 101, the stand-off portion 102, and the top portion of thermoacoustic probe housing 118 (the bottom portion is not attached (shown) in FIG. 5, enabling the view of the interior components of the thermoacoustic device 100.

FIG. 6 shows a perspective-front view of a the thermoacoustic device 100 without a stand-off or set-back portion. Shown are the thermoacoustic device 100, the radio-frequency (RF) emitter 101, the thermoacoustic transducer 120 and the thermoacoustic probe housing 118.

Although embodiments have been described above with reference to the accompanying drawings, those of skill in the art will appreciate that variations and modifications may be made without departing from the scope thereof as defined by the appended claims.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not to be exhaustive or to limit the embodiments to the precise forms disclosed. It will be apparent to one of ordinary skill in the art that many modifications and variations are possible in view of the above teachings.

What is claimed is:

1. Thermoacoustic imaging device for coupling to a region of interest on a patient, the device comprising:
a housing having a surface for coupling to the region of interest, wherein the surface comprises an acoustic coupling portion having a substantially perpendicular extent relative to the surface.

2. The thermoacoustic imaging device of claim 1, wherein the perpendicular extent of the acoustic coupling portion extends outwardly from the surface.

3. The thermoacoustic imaging device of claim 1, wherein the perpendicular extent of the acoustic coupling portion extends inwardly from the surface.

4. The thermoacoustic imaging device of claim 1, wherein the perpendicular extent of the acoustic coupling portion extends inwardly and outwardly from the surface.

5. The thermoacoustic imaging device of claim 3, wherein the perpendicular extent of the acoustic coupling portion extends inwardly from the surface forms a void space.

6. The thermoacoustic imaging device of claim 1, wherein the surface is formed of a first material and the acoustic matching portion is formed of a second material.

7. The thermoacoustic imaging device of claim 6, wherein the second material is a silicon-based elastomer.

8. The thermoacoustic imaging device of claim 6, wherein the second material is a silicon-based rubber.

10. The thermoacoustic imaging device of claim 6, wherein the second material is a silicon-based material.

11. The thermoacoustic imaging device of claim 1, further comprising:
a radio-frequency emitter comprising an exit window; and
wherein the thermoacoustic transducer further comprises a front face for receiving reflected acoustic energy; and
wherein the housing is configured to contain the thermoacoustic transducer and the radio-frequency emitter.

12. The thermoacoustic imaging device of claim 11, wherein the acoustic coupling portion has an electrical impedance outside of a range of 2 kilo-ohms to 5 kilo-ohms when the radio-frequency emitter provides pulsed radio-frequency energy between a range of 13 MHz and 2.54 Ghz.

13. The thermoacoustic device of claim 11, wherein a speed of sound in the acoustic coupling portion is between 1520 and 1560 meters per second.

14. Thermoacoustic imaging device for coupling to a region of interest on a patient, the device comprising:
a thermoacoustic transducer; and
a housing for containing the thermoacoustic transducer; and
wherein the housing comprises a surface, a portion of the surface having a portion for coupling to the region of interest, wherein the surface comprises an acoustic coupling portion having a substantially perpendicular extent relative to the thermoacoustic transducer.

15. The thermoacoustic imaging device of claim 14, wherein the perpendicular extent of the acoustic coupling portion extends outwardly from an end of the thermoacoustic transducer configured to receive acoustic energy.

16. The thermoacoustic imaging device of claim 15, wherein the perpendicular extent of the acoustic coupling portion extends to the surface.

17. The thermoacoustic imaging device of claim 16, wherein the perpendicular extent of the acoustic coupling portion further extends outwardly from the surface.

18. The thermoacoustic imaging device of claim 15, wherein the perpendicular extent of the acoustic coupling portion extends outwardly from the surface.

19. The thermoacoustic imaging device of claim 16, wherein the perpendicular extent of the acoustic coupling portion forms a void space.

20. The thermoacoustic imaging device of claim 14, wherein the surface is formed of a first material and the acoustic matching portion is formed of a second material.

21. The thermoacoustic imaging device of claim 20, wherein the second material is a silicon-based elastomer.

22. The thermoacoustic imaging device of claim 20, wherein the second material is a silicon-based rubber.

23. The thermoacoustic imaging device of claim 20, wherein the second material is a silicon-based material.

24. The thermoacoustic imaging device of claim 14, further comprising:
a radio-frequency emitter comprising an exit window; and
wherein the thermoacoustic transducer further comprises a front face for receiving reflected acoustic energy; and
wherein the housing is configured to contain the thermoacoustic transducer and the radio-frequency emitter.

25. The thermoacoustic imaging device of claim 24, wherein the acoustic coupling portion has an electrical impedance outside of a range of 2 kilo-ohms to 5 kilo-ohms when the radio-frequency emitter provides pulsed radio-frequency energy between a range of 13 MHz and 2.54 Ghz.

26. The thermoacoustic device of claim 24, wherein a speed of sound in the acoustic coupling portion is between 1520 and 1560 meters per second.

27. Thermoacoustic imaging device for coupling to a region of interest on a patient, the device comprising:
a radio-frequency emitter comprising an exit window;
a thermoacoustic transducer comprising a front face for receiving reflected acoustic energy;
a housing configured to contain the thermoacoustic transducer and the radio-frequency emitter, wherein the housing comprises a surface, a portion of the surface having a substantially planar portion for coupling to the region of interest, wherein the exit window of the radio-frequency emitter and front face of the thermoacoustic transducer are aligned flush with the surface; and
wherein the surface comprises an acoustic coupling portion having a substantially perpendicular extent relative to the front face of the thermoacoustic transducer.

28. The thermoacoustic imaging device of claim 27, wherein the acoustic coupling portion has an electrical impedance outside of a range of 2 kilo-ohms to 5 kilo-ohms when the radio-frequency emitter provides pulsed radio-frequency energy between a range of 13 MHz and 2.54 Ghz.

29. The thermoacoustic device of claim 27, wherein a speed of sound in the acoustic coupling portion is between 1520 and 1560 meters per second.

30. The thermoacoustic imaging device of claim 27, wherein the perpendicular extent of the acoustic coupling portion extends outwardly from the front face.

31. The thermoacoustic imaging device of claim 30, wherein the perpendicular extent of the front face extends to the surface.

32. The thermoacoustic imaging device of claim 31, wherein the perpendicular extent of the front face further extends outwardly from the surface.

33. The thermoacoustic imaging device of claim 30, wherein the perpendicular extent of the acoustic coupling portion extends outwardly from the surface.

34. The thermoacoustic imaging device of claim 31, wherein the perpendicular extent of the acoustic coupling portion forms a void space.

35. The thermoacoustic imaging device of claim 30, wherein the surface is formed of a first material and the acoustic matching portion is formed of a second material.

36. The thermoacoustic imaging device of claim 35, wherein the second material is a silicon-based elastomer.

37. The thermoacoustic imaging device of claim 35, wherein the second material is a silicon-based rubber.

38. The thermoacoustic imaging device of claim 35, wherein the second material is a silicon-based material.

39. The thermoacoustic imaging device of claim 35, wherein the exit window of the radio-frequency emitter and front face of the thermoacoustic transducer are aligned flush with the surface, and wherein the perpendicular extent of the acoustic coupling portion extends outwardly from the surface.

40. The thermoacoustic imaging device of claim 35, wherein the exit window of the radio-frequency emitter is aligned flush with the surface, and the front face of the thermoacoustic transducer is set-back from the surface, and wherein the perpendicular extent of the acoustic coupling portion extends outwardly from the front face to the surface.

41. The thermoacoustic imaging device of claim 40, wherein the perpendicular extent of the front face further extends outwardly from the surface.
